(19) United States
(12) Patent Application Publication
(10) Pub. No.: US 2023/0065493 A1
(43) Pub. Date: Mar. 2, 2023

(54) PROTEIN COMPOSITION WITH ISOPRENE POLYMERIZATION ACTIVITY AND APPLICATION THEREOF

(71) Applicant: Sumitomo Riko Company Limited, Komaki-shi (JP)

(72) Inventors: Shunsuke IMAI, Aichi (JP); Fuminori YONEYAMA, Aichi (JP); Osamu WAKISAKA, Aichi (JP)

(73) Assignee: Sumitomo Riko Company Limited, Komaki-shi (JP)

(21) Appl. No.: 17/968,818

(22) Filed: Oct. 19, 2022

Related U.S. Application Data
(63) Continuation of application No. PCT/JP2021/038921, filed on Oct. 21, 2021.

(30) Foreign Application Priority Data
Oct. 30, 2020 (JP) 2020-183088

(51) Int. Cl. C12P 5/00; C07K 14/415; C12N 15/82; C12N 9/10

(52) U.S. Cl. CPC C12P 5/007; C07K 14/415; C12N 15/8243; C12N 9/1085; B29K 2607/00

(57) ABSTRACT

The present disclosure provides a means for efficiently producing natural rubber and stably providing the same, and is aimed at stably providing a rubber resource. The present disclosure may provide a protein composition that exhibits an isoprene polymerization activity and comprises a protein(s) (B) exhibiting the same activity as CPTL and either one of proteins (A-1) exhibiting the same activity as CPT6 or (A-2) exhibiting the same activity as CPT7, as well as a lipid membrane structure comprising the composition, a method for producing the same, a cell expressing a protein constituting the composition, a method for producing the same, and a method for producing an isoprene polymer compound using any of the above.

### CROSS-REFERENCE TO RELATED APPLICATIONS

This application is a continuation of International Application No. PCT/JP2021/038921, filed Oct. 21, 2021, and claims priority therethrough under 35 U.S.C. § 119 to Japanese Patent Application No. 2020-183088, filed Oct. 30, 2020, each are incorporated herein by reference in its entirety.

### FIELD

This disclosure relates to protein compositions having isoprene polymerization activity and uses thereof, and in particular, to protein compositions having isoprene polymerization activity, lipid membrane structures and cells containing the same, and methods of producing isoprene polymer compounds using the same.

### BACKGROUND

Natural rubber is a substance composed mainly of cis-polyisoprene contained in sap of the rubber tree. It is widely used in various fields such as the automotive and construction industries, and its demand is expanding worldwide. Since natural rubber is an agricultural crop collected from rubber trees, its supply is unstable, and there are problems such as inconsistent quality, potentially causing a serious risk in business. For this reason, attempts are being made to develop technology to artificially produce natural rubber.

Patent Literature 1 and Non-Patent Literature 1 describe methods of producing polyisoprenoids. Each of the method involves a binding process of binding a rubber particle to each of gene expression products of predetermined proteins (HRT1, HRTBP and REF) involved in natural rubber biosynthesis in vitro.

### CITATION LIST

### Patent Literature

[Patent Literature 1] Japanese Patent No. 6586693

### Non Patent Literature

[Non-Patent Literature 1] Yamashita S. et al. eLife 2016; 5: e19022. doi: 10.7554/eLife.19022

### SUMMARY

### Technical Problems

The arts described in Patent Literature 1 and Non-Patent Literature 1 were insufficient to produce enough amount of polyisoprenoids for practical use. According to this technology, it is necessary to collect rubber particles from plants, which makes the process of collecting rubber particles complicated, and it is difficult to stabilize the quality of rubber particles because they are extracted from plants.

Some embodiments of the present disclosure provide a means for efficiently producing natural rubber and stably providing the same, and is aimed at stably providing a rubber resource.

### Solution to Problems

[1] A protein composition that exhibits an isoprene polymerization activity, the protein composition comprising a protein(s) (B) and either one of proteins (A-1) and (A-2):

(A-1) one or more protein(s) selected from a protein(s) comprising an amino acid sequence of SEQ ID NO: 6 and a protein(s) that comprises an amino acid sequence having 90% or more identity with the amino acid sequence of SEQ ID NO: 6 and exhibits the same activity as CPT6;

(A-2) one or more protein(s) selected from a protein(s) comprising an amino acid sequence of SEQ ID NO: 8 and a protein(s) that comprises an amino acid sequence having 90% or more identity with the amino acid sequence of SEQ ID NO: 8 and exhibits the same activity as CPT7; and

(B) one or more protein(s) selected from a protein(s) comprising an amino acid sequence of SEQ ID NO: 10 and a protein(s) that comprises an amino acid sequence having 90% or more identity with the amino acid sequence of SEQ ID NO: 10 and exhibits the same activity as CPTL.

[2] The composition according to [1], wherein the composition fulfills at least one of the followings:

the protein (A-1) encoded by one or more polynucleotide(s) (a-1) selected from a polynucleotide(s) comprising a nucleic acid sequence of SEQ ID NO: 5 and a polynucleotide(s) that comprises a nucleic acid sequence having 90% or more identity with the nucleic acid sequence of SEQ ID NO: 5 and encodes a protein exhibiting the same activity as CPT6;

the protein (A-2) encoded by one or more polynucleotide(s) (a-2) selected from a polynucleotide(s) comprising a nucleic acid sequence of SEQ ID NO: 7 and a polynucleotide(s) that comprises a nucleic acid sequence having 90% or more identity with the nucleic acid sequence of SEQ ID NO: 7 and encodes a protein exhibiting the same activity as CPT7; and

the protein (B) encoded by one or more polynucleotide(s) (b) selected from a polynucleotide(s) comprising a nucleic acid sequence of SEQ ID NO: 9 and a polynucleotide(s) that comprises a nucleic acid sequence having 90% or more identity with the nucleic acid sequence of SEQ ID NO: 9 and encodes a protein exhibiting the same activity as CPTL.

[3] The composition according to [1] or [2], further comprising an REF family protein, an SRPP family protein, and a CPT family protein other than CPT6, 7 and CPTL.
[4] The composition according to [1], further comprising at least one of the followings:

(C) one or more protein(s) selected from a protein(s) comprising an amino acid sequence of SEQ ID NO: 2 and a protein(s) that comprises an amino acid sequence having 90% or more identity with the amino acid sequence of SEQ ID NO: 2 and exhibits the same activity as CPT1;

(D) one or more protein(s) selected from a protein(s) comprising an amino acid sequence of SEQ ID NO: 4 and a protein(s) that comprises an amino acid sequence having 90% or more identity with the amino acid sequence of SEQ ID NO: 4 and exhibits the same activity as CPT2;

(E) one or more protein(s) selected from a protein(s) comprising an amino acid sequence of SEQ ID NO: 12 and a protein(s) that comprises an amino acid sequence having 90% or more identity with the amino acid sequence of SEQ ID NO: 12 and exhibits the same activity as REF1;

(F) one or more protein(s) selected from a protein(s) comprising an amino acid sequence of SEQ ID NO: 14 and a protein(s) that comprises an amino acid sequence having 90% or more identity with the amino acid sequence of SEQ ID NO: 14 and exhibits the same activity as REF2;

(G) one or more protein(s) selected from a protein(s) comprising an amino acid sequence of SEQ ID NO: 16 and a protein(s) that comprises an amino acid sequence having 90% or more identity with the amino acid sequence of SEQ ID NO: 16 and exhibits the same activity as REF8; and

(H) one or more protein(s) selected from a protein(s) comprising an amino acid sequence of SEQ ID NO: 18 and a protein(s) that comprises an amino acid sequence having 90% or more identity with the amino acid sequence of SEQ ID NO: 18 and exhibits the same activity as SRPP1.

[5] The composition according to [4], wherein the composition fulfills at least one of the followings:

the protein (C) encoded by one or more polynucleotide(s) (c) selected from a polynucleotide(s) comprising a nucleic acid sequence of SEQ ID NO: 1 and a polynucleotide(s) that comprises a nucleic acid sequence having 90% or more identity with the nucleic acid sequence of SEQ ID NO: 1 and encodes a protein exhibiting the same activity as CPT1;

the protein (D) encoded by one or more polynucleotide(s) (d) selected from a polynucleotide(s) comprising a nucleic acid sequence of SEQ ID NO: 3 and a polynucleotide(s) that comprises a nucleic acid sequence having 90% or more identity with the nucleic acid sequence of SEQ ID NO: 3 and encodes a protein exhibiting the same activity as CPT2;

the protein (E) encoded by one or more polynucleotide(s) (e) selected from a polynucleotide(s) comprising a nucleic acid sequence of SEQ ID NO: 11 and a polynucleotide(s) that comprises a nucleic acid sequence having 90% or more identity with the nucleic acid sequence of SEQ ID NO: 11 and encodes a protein exhibiting the same activity as REF1;

the protein (F) encoded by one or more polynucleotide(s) (f) selected from a polynucleotide(s) comprising a nucleic acid sequence of SEQ ID NO: 13 and a polynucleotide(s) that comprises a nucleic acid sequence having 90% or more identity with the nucleic acid sequence of SEQ ID NO: 13 and encodes a protein exhibiting the same activity as REF2;

the protein (G) encoded by one or more polynucleotide(s) (g) selected from a polynucleotide(s) comprising a nucleic acid sequence of SEQ ID NO: 15 and a polynucleotide(s) that comprises a nucleic acid sequence having 90% or more identity with the nucleic acid sequence of SEQ ID NO: 15 and encodes a protein exhibiting the same activity as REF8; and

the protein (H) encoded by one or more polynucleotide(s) (h) selected from a polynucleotide(s) comprising a nucleic acid sequence of SEQ ID NO: 17 and a polynucleotide(s) that comprises a nucleic acid sequence having 90% or more identity with the nucleic acid sequence of SEQ ID NO: 17 and encodes a protein exhibiting the same activity as SRPP1.

[6] The composition according to any one of [1] to [5], wherein at least one of the proteins (A) through (H) is a protein derived from a rubber tree.
[7] A lipid membrane structure comprising the composition according to any one of [1] to [6] and a phospholipid.
[8] The structure according to [7], wherein the structure is a lipid bilayered membrane structure.
[9] The structure according to [7] or [8], wherein the structure is a proteoliposome.
[10] A method for producing the lipid membrane structure according to any one of [7] to [9], the method comprising expressing the polynucleotide encoding the protein constituting the composition according to any one of [1] to [6] in a cell-free protein production system in the presence of a raw material containing a phospholipid.
[11] The method for producing the lipid membrane structure according to [10], wherein the polynucleotide encoding the protein constituting the composition according to any one of [1] to [6] comprises (b) and either one of (a-1) and (a-2):

(a-1) one or more polynucleotide(s) selected from a polynucleotide(s) comprising a nucleic acid sequence of SEQ ID NO: 5 and a polynucleotide(s) that comprises a nucleic acid sequence having 90% or more identity with the nucleic acid sequence of SEQ ID NO: 5 and encodes a protein exhibiting the same activity as CPT6;

(a-2) one or more polynucleotide(s) selected from a polynucleotide(s) comprising a nucleic acid sequence of SEQ ID NO: 7 and a polynucleotide(s) that comprises a nucleic acid sequence having 90% or more identity with the nucleic acid sequence of SEQ ID NO: 7 and encodes a protein exhibiting the same activity as CPT7; and

(b) one or more polynucleotide(s) selected from a polynucleotide(s) comprising a nucleic acid sequence of SEQ ID NO: 9 and a polynucleotide(s) that comprises a nucleic acid sequence having 90% or more identity with the nucleic acid sequence of SEQ ID NO: 9 and encodes a protein exhibiting the same activity as CPTL.

[12] The method for producing the lipid membrane structure according to [10] or [11], wherein the polynucleotide is the polynucleotide encoding the protein constituting the composition according to [3] or [4], and comprises at least one of the followings:

(c) one or more polynucleotide(s) selected from a polynucleotide(s) comprising a nucleic acid sequence of SEQ ID NO: 1 and a polynucleotide(s) that comprises a nucleic acid sequence having 90% or more identity with the nucleic acid sequence of SEQ ID NO: 1 and encodes a protein exhibiting the same activity as CPT1;

(d) one or more polynucleotide(s) selected from a polynucleotide(s) comprising a nucleic acid sequence of SEQ ID NO: 3 and a polynucleotide(s) that comprises a nucleic acid sequence having 90% or more identity with the nucleic acid sequence of SEQ ID NO: 3 and encodes a protein exhibiting the same activity as CPT2;

(e) one or more polynucleotide(s) selected from a polynucleotide(s) comprising a nucleic acid sequence of SEQ ID NO: 11 and a polynucleotide(s) that comprises a nucleic acid sequence having 90% or more identity with the nucleic acid sequence of SEQ ID NO: 11 and encodes a protein exhibiting the same activity as REF1;

(f) one or more polynucleotide(s) selected from a polynucleotide(s) comprising a nucleic acid sequence of SEQ ID NO: 13 and a polynucleotide(s) that comprises a nucleic acid sequence having 90% or more identity with the nucleic acid sequence of SEQ ID NO: 13 and encodes a protein exhibiting the same activity as REF2;

(g) one or more polynucleotide(s) selected from a polynucleotide(s) comprising a nucleic acid sequence of SEQ ID NO: 15 and a polynucleotide(s) that comprises a nucleic acid sequence having 90% or more identity with the nucleic acid sequence of SEQ ID NO: 15 and encodes a protein exhibiting the same activity as REF8; and

(h) one or more polynucleotide(s) selected from a polynucleotide(s) comprising a nucleic acid sequence of SEQ ID NO: 17 and a polynucleotide(s) that comprises a nucleic acid sequence having 90% or more identity with the nucleic acid sequence of SEQ ID NO: 17 and encodes a protein exhibiting the same activity as SRPP1.

[13] The method for producing the lipid membrane structure according to any one of [10] to [12], wherein the raw material containing the phospholipid contains a liposome, and wherein the lipid membrane structure is a proteoliposome.
[14] A cell expressing a protein constituting the composition of any one of [1] to [5].
[15] The cell according to [14], comprising an expression unit containing a polynucleotide that encodes each of the proteins in the protein composition of any one of [1] to [6].
[16] The cell according to [14] or [15], wherein the polynucleotide comprises (b) and either one of (a-1) and (a-2):

(a-1) one or more polynucleotide(s) selected from a polynucleotide(s) comprising a nucleic acid sequence of SEQ ID NO: 5 and a polynucleotide(s) that comprises a nucleic acid sequence having 90% or more identity with the nucleic acid sequence of SEQ ID NO: 5 and encodes a protein exhibiting the same activity as CPT6;

(a-2) one or more polynucleotide(s) selected from a polynucleotide(s) comprising a nucleic acid sequence of SEQ ID NO: 7 and a polynucleotide(s) that comprises a nucleic acid sequence having 90% or more identity with the nucleic acid sequence of SEQ ID NO: 7 and encodes a protein exhibiting the same activity as CPT7; and

(b) one or more polynucleotide(s) selected from a polynucleotide(s) comprising a nucleic acid sequence of SEQ ID NO: 9 and a polynucleotide(s) that comprises a nucleic acid sequence having 90% or more identity with the nucleic acid sequence of SEQ ID NO: 9 and encodes a protein exhibiting the same activity as CPTL.

[17] The cell according to any one of [14] to [16], wherein the polynucleotide is the polynucleotide encoding the protein constituting the composition according to [3] or [4], and further comprises at least one of the followings:

(c) one or more polynucleotide(s) selected from a polynucleotide(s) comprising a nucleic acid sequence of SEQ ID NO: 1 and a polynucleotide(s) that comprises a nucleic acid sequence having 90% or more identity with the nucleic acid sequence of SEQ ID NO: 1 and encodes a protein exhibiting the same activity as CPT1;

(d) one or more polynucleotide(s) selected from a polynucleotide(s) comprising a nucleic acid sequence of SEQ ID NO: 3 and a polynucleotide(s) that comprises a nucleic acid sequence having 90% or more identity with the nucleic acid sequence of SEQ ID NO: 3 and encodes a protein exhibiting the same activity as CPT2;

(e) one or more polynucleotide(s) selected from a polynucleotide(s) comprising a nucleic acid sequence of SEQ ID NO: 11 and a polynucleotide(s) that comprises a nucleic acid sequence having 90% or more identity with the nucleic acid sequence of SEQ ID NO: 11 and encodes a protein exhibiting the same activity as REF1;

(f) one or more polynucleotide(s) selected from a polynucleotide(s) comprising a nucleic acid sequence of SEQ ID NO: 13 and a polynucleotide(s) that comprises a nucleic acid sequence having 90% or more identity with the nucleic acid sequence of SEQ ID NO: 13 and encodes a protein exhibiting the same activity as REF2;

(g) one or more polynucleotide(s) selected from a polynucleotide(s) comprising a nucleic acid sequence of SEQ ID NO: 15 and a polynucleotide(s) that comprises a nucleic acid sequence having 90% or more identity with the nucleic acid sequence of SEQ ID NO: 15 and encodes a protein exhibiting the same activity as REF8; and

(h) one or more polynucleotide(s) selected from a polynucleotide(s) comprising a nucleic acid sequence of SEQ ID NO: 17 and a polynucleotide(s) that comprises a nucleic acid sequence having 90% or more identity with the nucleic acid sequence of SEQ ID NO: 17 and encodes a protein exhibiting the same activity as SRPP1.

[18] The cell according to any one of [14] to [17], wherein the expression unit is a heterologous expression unit.
[19] The cell according to [14], comprising the lipid membrane structure according to [9].
[20] The method for producing the cell according to [14], comprising interacting the cell with the lipid membrane structure according to [9].
[21] A method for producing an isoprene polymer compound, the method comprising performing an isoprene polymerization reaction using at least one selected from the composition according to any one of [1] to [6], the structure according to any one of [7] to [9], and the cell according to any one of [14] to [19].
[22] The production method according to [21], wherein the isoprene polymerization reaction is performed using a low molecular weight allyl compound as a substrate.
[23] A kit for producing an isoprene polymer compound, the kit comprising:

a low molecular weight allyl compound; and

at least one selected from the composition according to any one of [1] to [6], the structure according to any one of [7] to [9], and the cell according to any one of [14] to [19].

[24] A method for producing a rubber, the method comprising producing the rubber using an isoprene polymer compound produced by the production method according to [21] or [22].

### Advantageous Effects of Disclosure

Some embodiments of the present disclosure are expected to efficiently produce and stably provide natural rubber, thereby stably providing a rubber resource for which demand is growing worldwide.

### DESCRIPTION OF EMBODIMENTS

[Protein Composition]

### (Isoprene Polymerization Activity)

A protein composition according to one embodiment of the present disclosure exhibits isoprene polymerization activity. The isoprene polymerization activity typically refers to an activity that directly or indirectly promotes an isoprene polymerization reaction to produce an isoprene polymer compound. The isoprene polymerization reaction is typically a polymerization reaction of isoprene in living organisms. Examples of substrates include low molecular weight allyl compounds such as isopentenyl diphosphate (IPP), farnesyl diphosphate (FPP), dimethylallyl diphosphate (DMAPP), geranyl diphosphate (GPP) and geranylgeranyl diphosphate (GGPP).

Examples of the organisms include rubber trees (plants capable of producing natural rubber) such as plants of the genus Hevea (e.g., para rubber tree (Hevea brasiliensis), Hevea benthamiana, Hevea guianensis), plants of the genus Ficus (e.g., Indian rubber tree (Ficus elastica), oak rubber tree (Ficus lyrata) and Benjamin rubber tree (Ficus benjamina), and the organism is not limited to the above plants. Examples of the organisms also include plant cells, animal cells and microorganisms.

(Protein Constituting Protein Composition)

### (Proteins (A) and (B))

The protein composition according to one embodiment of the present disclosure contains a protein(s) (B) and at least one of proteins (A-1) or (A-2):

(A-1) one or more protein(s) selected from a protein(s) comprising an amino acid sequence of SEQ ID NO: 6 and a protein(s) that comprises an amino acid sequence having 90% or more identity with the amino acid sequence of SEQ ID NO: 6 and exhibits the same activity as CPT6;

(A-2) one or more protein(s) selected from a protein(s) comprising an amino acid sequence of SEQ ID NO: 8 and a protein(s) that comprises an amino acid sequence having 90% or more identity with the amino acid sequence of SEQ ID NO: 8 and exhibits the same activity as CPT7; and

(B) one or more protein(s) selected from a protein(s) comprising an amino acid sequence of SEQ ID NO: 10 and a protein(s) that comprises an amino acid sequence having 90% or more identity with the amino acid sequence of SEQ ID NO: 10 and exhibits the same activity as CPTL.

The amino acid sequences of SEQ ID NO: 6, 8 and 10 are amino acid sequences of cis-prenyltransferase (CPT) 6, CPT7 and CPTL, respectively, derived from para rubber tree (Hevea brasiliensis).

As well, the proteins (A-1), (A-2) and (B) may be proteins encoded by polynucleotides (a-1), (a-2) and (b) encoding (A-1), (A-2) and (B), respectively:

(a-1) one or more polynucleotide(s) selected from a polynucleotide(s) comprising a nucleic acid sequence of SEQ ID NO: 5 and a polynucleotide(s) that comprises a nucleic acid sequence having 90% or more identity with the nucleic acid sequence of SEQ ID NO: 5 and encodes a protein exhibiting the same activity as CPT6;
(a-2) one or more polynucleotide(s) selected from a polynucleotide(s) comprising a nucleic acid sequence of SEQ ID NO: 7 and a polynucleotide(s) that comprises a nucleic acid sequence having 90% or more identity with the nucleic acid sequence of SEQ ID NO: 7 and encodes a protein exhibiting the same activity as CPT7; and
(b) one or more polynucleotide(s) selected from a polynucleotide(s) comprising a nucleic acid sequence of SEQ ID NO: 9 and a polynucleotide(s) that comprises a nucleic acid sequence having 90% or more identity with the nucleic acid sequence of SEQ ID NO: 9 and encodes a protein exhibiting the same activity as CPTL.

The nucleic acid sequences of SEQ ID NO: 5, 7 and 9 are full-length nucleic acid sequences of CPT6, CPT7 and CPTL, respectively, derived from para rubber tree (Hevea brasiliensis).

(Other Proteins)

The protein composition may contain (an)other protein(s) in addition to the proteins (A-1), (A-2) and (B). Examples of the other protein(s) include proteins responsible for the isoprene polymerization reaction itself in vivo and proteins known to assist in the polymerization reaction. Examples of the former include CPT families other than CPT6, CPT7 and CPTL. Examples of the latter includes at least one selected from a rubber elongation factor (REF) family and a small rubber particle protein (SRPP) family. Among them, at least one of proteins (C) to (H) is preferred:

(C) one or more protein(s) selected from a protein(s) comprising an amino acid sequence of SEQ ID NO: 2 and a protein(s) that comprises an amino acid sequence having 90% or more identity with the amino acid sequence of SEQ ID NO: 2 and exhibits the same activity as CPT1;

(D) one or more protein(s) selected from a protein(s) comprising an amino acid sequence of SEQ ID NO: 4 and a protein(s) that comprises an amino acid sequence having 90% or more identity with the amino acid sequence of SEQ ID NO: 4 and exhibits the same activity as CPT2;

(E) one or more protein(s) selected from a protein(s) comprising an amino acid sequence of SEQ ID NO: 12 and a protein(s) that comprises an amino acid sequence having 90% or more identity with the amino acid sequence of SEQ ID NO: 12 and exhibits the same activity as REF1;

(F) one or more protein(s) selected from a protein(s) comprising an amino acid sequence of SEQ ID NO: 14 and a protein(s) that comprises an amino acid sequence having 90% or more identity with the amino acid sequence of SEQ ID NO: 14 and exhibits the same activity as REF2;

(G) one or more protein(s) selected from a protein(s) comprising an amino acid sequence of SEQ ID NO: 16 and a protein(s) that comprises an amino acid sequence having 90% or more identity with the amino acid sequence of SEQ ID NO: 16 and exhibits the same activity as REF8; and

(H) one or more protein(s) selected from a protein(s) comprising an amino acid sequence of SEQ ID NO: 18 and a protein(s) that comprises an amino acid sequence having 90% or more identity with the amino acid sequence of SEQ ID NO: 18 and exhibits the same activity as SRPP1.

The amino acid sequences of SEQ ID NO: 2, 4, 12, 14, 16 and 18 include mature proteins of CPT1, CPT2, REF1, REF2, REF8 and SRPP1, respectively, derived from para rubber tree (Hevea brasiliensis).

As well, the proteins (C) to (H) may be proteins encoded by polynucleotides (c) to (h) encoding (C) to (H), respectively:

(c) one or more polynucleotide(s) selected from a polynucleotide(s) comprising a nucleic acid sequence of SEQ ID NO: 1 and a polynucleotide(s) that comprises a nucleic acid sequence having 90% or more identity with the nucleic acid sequence of SEQ ID NO: 1 and encodes a protein exhibiting the same activity as CPT1;
(d) one or more polynucleotide(s) selected from a polynucleotide(s) comprising a nucleic acid sequence of SEQ ID NO: 3 and a polynucleotide(s) that comprises a nucleic acid sequence having 90% or more identity with the nucleic acid sequence of SEQ ID NO: 3 and encodes a protein exhibiting the same activity as CPT2;
(e) one or more polynucleotide(s) selected from a polynucleotide(s) comprising a nucleic acid sequence of SEQ ID NO: 11 and a polynucleotide(s) that comprises a nucleic acid sequence having 90% or more identity with the nucleic acid sequence of SEQ ID NO: 11 and encodes a protein exhibiting the same activity as REF1;
(f) one or more polynucleotide(s) selected from a polynucleotide(s) comprising a nucleic acid sequence of SEQ ID NO: 13 and a polynucleotide(s) that comprises a nucleic acid sequence having 90% or more identity with the nucleic acid sequence of SEQ ID NO: 13 and encodes a protein exhibiting the same activity as REF2;
(g) one or more polynucleotide(s) selected from a polynucleotide(s) comprising a nucleic acid sequence of SEQ ID NO: 15 and a polynucleotide(s) that comprises a nucleic acid sequence having 90% or more identity with the nucleic acid sequence of SEQ ID NO: 15 and encodes a protein exhibiting the same activity as REF8; and
(h) one or more polynucleotide(s) selected from a polynucleotide(s) comprising a nucleic acid sequence of SEQ ID NO: 17 and a polynucleotide(s) that comprises a nucleic acid sequence having 90% or more identity with the nucleic acid sequence of SEQ ID NO: 17 and encodes a protein exhibiting the same activity as SRPP1.

The nucleic acid sequences of SEQ ID NO: 1, 3, 11, 13, 15 and 17 are full-length nucleic acid sequences of CPT1, CPT2, REF1, REF2, REF8 and SRPP1, respectively, derived from para rubber tree (Hevea brasiliensis).

The protein composition preferably contains two, three, four or five or more of the proteins (C) through (H), more preferably contains a combination of at least one of (C) and (D) and at least one of (E) through (H), and still more preferably contains all.

(Identity %)

The sequence identities of amino acid and nucleotide may be 90%, 91%, 92%, 93%, 94%, 95%, 96%, 97%, 98% or 99% or more. The identity can be determined under default conditions using NCBI BLAST (see http://www.ncbi.nlm.nih.gov), for example.

(Activity Possessed by Mutant Protein)

The term “exhibiting same activity” as each protein means exhibiting 50% or more, 60% or more, 70% or more, 80% or more, 90% or more, 95% or more, or 100% of the activity of each protein measured under the same condition.

(Mutation)

The above proteins may have mutations introduced at a site in a catalytic domain and at a site outside the catalytic domain, as long as they maintain same activity. A person skilled in the art can determine the position of the amino acid residue to which the mutation may be introduced in the protein while maintaining the aimed activity. For example, a person skilled in the art can 1) compare the amino acid sequences (e.g., the amino acid sequence represented by SEQ ID NO: 6 or 8, and the amino acid sequences of other proteins involved in the isoprene polymerization reaction) of a plurality of proteins having similar activities, 2) identify a relatively conserved region(s) and a region(s) not conserved, and then 3) predict regions capable and incapable of serving important roles in function from the relatively conserved region(s) and the region(s) not conserved in order to recognize the correlation in structure and/or function and determine a portion(s) to which the mutation can be introduced.

The mutation introduced into the protein can be a substitution of an amino acid residue for another amino acid residue, preferably a substitution with an amino acid residue having a similar side chain. Examples of classification of amino acids by amino acid residues with similar side chains include: amino acids with basic side chains such as lysine, arginine and histidine; amino acids with acidic side chains such as aspartic acid and glutamic acid; amino acids with uncharged polar side chains such as asparagine, glutamine, serine, threonine, tyrosine and cysteine; amino acids with nonpolarized side chains such as glycine, alanine, valine, leucine, isoleucine, proline, phenylalanine, methionine and tryptophan; amino acids with beta-branched side chains such as leucine, valine and isoleucine; amino acids with aromatic side chains such as tyrosine and phenylalanine, tryptophan and histidine; amino acids with hydroxyl group-containing side chains (e.g., alcohol, phenoxy group-containing side chains) such as serine, threonine and tyrosine; and amino acids with sulfur-containing side chains such as cysteine and methionine.

The nucleic acid sequences may be modified for codon optimization. The codon optimization in this description refers to modification involving optimization of an expression in a targeted certain cell without changing the sequence of the encoded polypeptide. A person skilled in the art can understand a frequency of codon usage in the cell to be transduced and easily determine the sequence obtained after optimization by utilizing genetic code degeneracy. Examples of the target cells include microorganisms such as E. coli, plant cells and animal cells.

(Protein Modification)

Each of the proteins constituting the protein composition may have a purification tag at the C-terminus. Examples of the purification tag include histidine tags; HA, FLAG, V5 and myc epitopes; chitin binding protein (CBP); maltose binding protein (MBP); glutathione-S-transferase (GST); and Strep tags. The protein composition may contain other factors directly or indirectly involved in the isoprene polymerization reaction in addition to those listed above.

(Lipid Membrane Structure)

The lipid membrane structure of the present disclosure may be a structure including lipid membranes that contains the above protein composition. The use of the lipid membrane structure may enable the protein composition to facilitate the isoprene polymerization activity, and achieve efficient isoprene polymerization.

The lipid membrane can be a monolayered membrane or a bilayered membrane. Examples of the structure of the monolayered membrane include rubber particles (particles extracted from latex derived from the rubber tree). Examples of the structure of the bilayered membrane include proteoliposomes and artificial lipid membrane models. The artificial lipid membrane models may be composed of either one or both of natural lipids and synthetic lipids, and are not particularly limited. The lipid membrane structure is preferably the structure of the bilayered membrane, more preferably proteoliposome.

(Proteoliposome)

In this description, proteoliposome refers to an artificial vesicle in which a protein is encapsulated or bound to a portion of the lipid bilayer of liposome. In this description, liposome refers to an artificial vesicle in which an aqueous component (usually water) is trapped in a space formed by the lipid bilayer membrane. The lipid bilayer membrane is typically composed of a lipid(s) such as phospholipid. Example of the phospholipid include phosphatidylethanolamine, phosphatidylcholine, phosphatidylserine, cardiolipin, or combinations thereof. Example of the lipid other than phospholipid include triacylglycerol, waxes, sphingolipids and sterols and fatty acid esters thereof, or combinations thereof. The lipid constituting the lipid bilayer membrane preferably include a biogenic phospholipid, more preferably include soybean-derived phospholipid.

The size of liposome is not particularly limited, but typically 50 to 300 nm in diameter. Liposome may be naturally occurring or synthesized, and is preferably the former, more preferably liposome derived from a plant such as soybean. The synthesized liposome may be polyalkylene glycol-based liposome, for example.

The form of the protein composition in the membrane lipid structure is not particularly limited, but presumed to be a form in which the protein composition is bound to hydrophilic groups of the phospholipids on the liposome surface (some may be embedded in the membrane interior) in the case of liposome.

(Production Method)

A method for producing the membrane lipid structure may be a method involving expression of the polynucleotide encoding the protein constituting the composition in a cell-free protein production system in the presence of source materials including the phospholipids, for example.

The source materials including phospholipids include the liposome if the membrane structure is the proteoliposome. The liposome is preferably the naturally occurring liposome, as described above, and preferably the liposome derived from the plant such as soybean.

The method for producing the proteoliposome preferably includes expressing an expression unit containing the polynucleotide in the cell-free protein production system in the presence of liposome.

The expression unit contains at least polynucleotides encoding the proteins contained in the protein composition. The polynucleotides refer to polynucleotides encoding the proteins constituting the protein composition, include (b) and either one of (a-1) and (a-2) described above, and further include at least one of (c) through (h) as required. The expression unit typically includes a promoter. This allows a polynucleotide expression product encoded by the polynucleotides to be efficiently produced. Examples of the promoter include tac promoter, lac promoter, trp promoter, trc promoter, T7 promoter, T5 promoter, T3 promoter, and SP6 promoter, and may include an enhancer. The promoter is linked to the polynucleotide in a form capable of expressing it. For example, promoter can be linked in an upstream (5′) side of the code sequence. The expression unit can be DNA or RNA, but is preferably DNA. The expression unit may contain factors such as terminators, ribosome binding sites, drug resistance genes, RNA processing signals, sequences that improve translation efficiency, sequences that improve protein stability, and sequences that improve protein secretion.

The cell-free protein production system is preferably performed using a cell extract (e.g., wheat germ extract, E. coli extract, rabbit reticulocyte extract and insect cell extract) by a bilayer-dialysis method and can be performed using a protein cell-free expression kit commercially available.

It is possible to use another protein building system, such as artificial lipid membrane model, instead of the liposome. The protein composition in the artificial lipid membrane model can be formed according to a conventional method. The artificial lipid membrane model may be composed of either one or both of naturally occurring lipids, synthetic lipids, and is not particularly limited.

(Cell)

The cell of the present disclosure may be a cell that expresses each of the proteins that make up the protein composition (proteins (A) and (B), and at least one protein of (C) through (H), as required). Examples of the cell include cells of microorganisms such as E. coli and yeast, plant cells, and animal cells.

The cell preferably contains the expression unit containing the polynucleotide(s). The expression unit is same as those explained for the proteoliposome. The expression unit containable in cells is preferably a heterologous expression unit. The heterologous expression unit in this description refers to an expression unit in which at least one of the polynucleotides encoding each of the proteins and the promoter normally contained is not intrinsic to the host cell. Preferably, both are not intrinsic to the host cell.

A method for producing the cell may be a method of transforming with an expression vector containing an expression unit that contains a polynucleotide encoding each of the proteins constituting the above protein compositions, for example. Since the protein composition contains at least two proteins as described above, the expression vector may be one (containing an expression unit containing polynucleotides encoding all proteins) or two or more (containing an expression unit containing polynucleotides for each or every few). The expression vector may be an integrative vector or a non-integrative vector. In the expression vector, the polynucleotide(s) can be placed under the control of a constitutive promoter or an inducible promoter.

A host can be transformed with the expression vector by one or more well-known method(s). Examples of such methods include calcium phosphate method, liposome method, DEAE dextran method, electroporation method, and particle gun (gene gun) method. The transformation can be performed by a method of infecting for the introduction in bacteria cells with use of a phage vector in addition to the plasmid vector.

The cell may be a cell containing the proteoliposome described above, as well. The proteoliposome is preferably prepared from the naturally occurring liposome. This facilitates interaction with the cell membrane. The cell containing proteoliposome can be produced by interacting the proteoliposome with the cell membrane. Examples of the methods for the interaction include adsorption or binding of the liposome to the cell surface, uptake of the liposome into the cell (endocytosis or phagocytosis), and fusion of the lipid bilayer membrane of the liposome with the cell membrane.

[Method for Producing Isoprene Polymerization Compound]

The above protein composition, the proteoliposome and the cells can be used to produce the isoprene polymerization compound.

In the production of isoprene polymerization compound, a low molecular weight allylic compound is used as a substrate to perform the isoprene polymerization reaction. Examples of the low molecular weight allylic compound include isoprenyl diphosphate and farnesyl diphosphate, both of which are preferably used. Conditions such as reaction temperature and reaction time can be set as appropriate. A typical isoprene polymerization compound is cis-polyisoprene, which is useful as a raw material of the rubber. Other examples of the isoprene polymerization compound include dolichol, polyprenol and derivatives thereof, which exhibit physiological activities (e.g., immune activity, antiviral activity and antioxidant activity) in the body and are therefore useful as materials for pharmaceuticals and health foods. It can be used as an additive (e.g., plasticizer, compatibilizer) for rubber products, as well.

[Kit for the Production of Isoprene Polymerization Compound]

The above protein composition, proteoliposome and the cells can be used as a kit for the production of isoprene polymerization compound. The Kit typically contains the low molecular weight allyl compound as the substrate. The Kit may further contain a reaction vessel and needed reagents.

[Method for Producing Rubber]

The isoprene polymer compound obtained by the above isoprene polymer compound production method can be used in the production of rubber. The procedure and conditions for producing the rubber from the isoprene polymer compound may be in conformity with the conventional method, such as a method of kneading the isoprene polymer compound, cross-linking and then molding, for example. Examples of the additives that can be used as required include reinforcing agents, silane coupling agents, fillers, vulcanizing agents, vulcanization accelerators, vulcanization accelerator auxiliary agents, oils, curing resins, waxes, anti-aging agents and coloring agents. These additives are added at appropriate stages. The resulting rubber can be used for various applications such as tires, construction materials, sporting goods, and automotive parts.

### EXAMPLES

### Examples 1 to 11

### (1) Acquisition of Natural Rubber Biosynthesis Protein Gene

Seven proteins (CPT1, CPT2, CPTL, REF1, REF2, REF8 and SRPP1) capable of being produced in latex at high yields were selected from “Rubber Database” (http://Matsui-lab.riken.jp/rubber/home.html). Two proteins (CPT6 and CPT7) were also selected from the phylogenetic analysis.

PCR was carried out using plasmids containing genes encoding the proteins or cDNA thereof as templates to provide genes of CPT1, CPT2, CPT6, CPT7, CPTL, REF1, REF2, REF8 and SRPP1. PCR was performed according to the instructions for PrimeSTAR Max DNA Polymerase (Takara Bio Inc.) or KOD One PCR Master Mix (TOYOBO CO., LTD.). A restriction enzyme digestion site and a His tag sequence were added to the primer as required.

The following primer sets were used for the acquisition of the genes of CPT1, CPT2, CPT6, CPT7, CPTL, REF1, REF2, REF8 and SRPP1: a set of primers 1 and 2, a set of primers 3 and 4, a set of primers 5 and 6, a set of primers 7 and 8, a set of primers 9 and 10, a set of primers 11 and 12, a set of primers 13 and 14, a set of primers 15 and 16, and a set of primers 17 and 18, respectively.

(Primer set for acquisition of CPT1 gene)
Primer 1:
(SEQ ID NO: 19)
5′-atggaattatacaacggtg-3′
Primer 2:
(SEQ ID NO: 20)
5′-atctcgagttaatgatgatgatgatgatgttttaag-3′
(Primer set for acquisition of CPT2 gene)
Primer 3:
(SEQ ID NO: 21)
5′-atggaaatatatacgggtcag-3′
Primer 4:
(SEQ ID NO: 22)
5′-atctcgagttaatgatgatgatgatgatgttttaaatattc-3′
(Primer set for acquisition of CPT6 gene)
Primer 5:
(SEQ ID NO: 23)
5′-atggaaaaacatagcagtag-3′
Primer 6:
(SEQ ID NO: 24)
5′-atctcgagttatataactgatgctttttc-3′
(Primer set for acquisition of CPT7 gene)
Primer 7:
(SEQ ID NO: 25)
5′-atgcaatccttgcacttg-3′
Primer 8:
(SEQ ID NO: 26)
5′-atctcgagttatgtaagtcgtctaccatag-3′
(Primer set for acquisition of CPTL gene)
Primer 9:
(SEQ ID NO: 27)
5′-atggatttgaaacctgg-3′
Primer 10:
(SEQ ID NO: 28)
5′-atctcgagttaatgatgatgatgatgatgtgtac-3′
(Primer set for acquisition of REF1 gene)
Primer 11:
(SEQ ID NO: 29)
5′-atggctgaaggtgaagaagaggtgaatatc-3′
Primer 12:
(SEQ ID NO: 30)
5′-atctcgagtcacccatctccatatagcac-3′
(Primer set for acquisition of REF2 gene)
Primer 13:
(SEQ ID NO: 31)
5′-atggctgaagacgaagacaaccaacaag-3′
Primer 14:
(SEQ ID NO: 32)
5′-atctcgagtcaattctctccataaaacac-3′
(Primer set for acquisition of REF8 gene)
Primer 15:
(SEQ ID NO 33)
5′-atggctgaagggaaagaaaacgagaatttc-3′
Primer 16:
(SEQ ID NO: 34)
5′-atctcgagttactctgcactttccttcac-3′
(Primer set for acquisition of SRPP1 gene)
Primer 17:
(SEQ ID NO: 35)
5′-atggctgaagaggtggaggaagagaggc-3′
Primer 18:
(SEQ ID NO: 36)
5′-atctcgagttatgatgcctcatctccaaac-3′

For each gene obtained by the method described above, the sequence was identified, and the full-length nucleic acid sequence and amino acid sequence were identified. SEQ ID NO: 1, 3, 5, 7, 9, 11, 13, 15 and 17 represent nucleic acid sequences of CPT1, CPT2, CPT6, CPT7, CPTL, REF1, REF2, REF8 and SRPP1 genes, respectively. SEQ ID NO: 2, 4, 6, 8, 10, 12, 14, 16 and 18 represent amino acid sequences of CPT1, CPT2, CPT6, CPT7, CPTL, REF1, REF2, REF8 and SRPP1 genes, respectively.

(2) Preparation of Plasmid for Cell-Free Protein Synthesis

Each of PCR reaction products of the genes obtained in the above (1) was treated with a restriction enzyme XhoI added to the primer. The combinations listed in Table 1 were selected to be inserted into a cell-free expression vector pEU-E01-MCS treated with EcoRV and XhoI restriction enzymes to prepare plasmids for cell-free protein synthesis.

(3) Transformation of E. coli

The prepared plasmids were transformed in E. coli JM 109. The transformant was cultured in LB agar medium containing ampicillin to select the transformant to which a target plasmid was introduced by colony PCR.

(4) Preparation of Plasmid for Cell-Free Protein Synthesis

E. coli transformed with a plasmid containing the target gene was cultured in LB liquid medium at 37° C. overnight. The bacterial cells were collected for recovery of the plasmid. Plasmids were recovered using the FastGene Plasmid Mini Kit (Nippon Genetics Co., Ltd.). The absence of mutations in the gene sequence inserted in the recovered plasmid was confirmed by sequencing analysis.

(5) Transformation of E. coli

The plasmid prepared as above was used for transformation of E. coli JM109. The transformant was cultured in LB agar medium containing ampicillin.

(6) Recovery of Plasmid for Cell-Free Protein Synthesis

E. coli transformed with a plasmid containing the target gene was cultured in LB liquid medium at 37° C. overnight. The bacterial cells were collected for recovery of the plasmid. The plasmid was recovered using QIAGEN Plasmid Midi Kit (QIAGEN) and prepared to 1 μg/μL.

(7) Synthesis of Membrane Protein (Proteoliposome) in the Presence of Liposome by Bilayer-Dialysis Method

A lipid bilayered membrane (liposome: approximately several hundreds nm in diameter) was prepared with use of soybean-derived phospholipids. Cell-free protein synthesis was performed under liposome addition conditions according to the protocol supplied with the ProteoLiposome BD Expression Kit (CellFree Sciences Co., Ltd.). The plasmids for expression of the nine proteins used as templates were prepared to 1 μg/μL and used according to the combinations listed in Table 1 in equivalent ratio so that the total volume was approximately 13 μL. For example, in Example 3, 1.5 μL for each of 9 species, 13.5 μL in total was added to the transcription reaction system. After the synthesis reaction was completed, a simple purification of Proteoliposomes with 9 proteins bound to liposomes was performed according to the protocol provided, and the synthesis was confirmed by SDS-PAGE.

(8) Measurement of Isoprene Polymerization Activity

The isoprene polymerization activities of cell-free synthesized proteins were determined by the following method.

Tris-HCl (pH 7.5) at a final concentration of 50 mM was mixed with MgCl₂ at a final concentration of 10 mM, DTT at a final concentration of 2 mM, farnesyl diphosphate (FPP) at a final concentration of 10 μM, ¹⁴C-isopentenyldiphosphate (¹⁴C-IPP) (specific activity: 1.48 to 2.22 GBq/mmol) at a final concentration of 50 μM, and 5 μL Proteoliposome to prepare 100 μL of reaction solution. The reaction solution was allowed to react at 30° C. for 24 hours.

After the reaction, 200 μL of saturated saline solution was added and the reaction product was extracted with 300 μL of water-saturated n-butanol. 400 μL of 2M NaCl was added to the collected water-saturated n-butanol layer for suspension to collect the water-saturated n-butanol layer. To 50 μL of the collected solution, 5 mL of Clear sol II (Nacalai Tesque, Inc.) was added and mixed well. The mixture solution was used for measurement of radioactivity (dpm) with a liquid scintillation counter (LSC-8000, Aloka Co., Ltd.). The reaction system without addition of Proteoliposome was used as a negative control. Table 1 represents results for Proteoliposome containing various proteins.

(9) TLC Radiogram of Isoprene Polymerization Reaction Product

An organic layer was volatilized from the water-saturated n-butanol solution collected as above with centrifugal evaporator. 1.5 mL of a reaction solution was prepared by mixing sodium acetate (pH 5.5) at a final concentration of 100 mM with acid phosphatase at a final concentration of 1 U and 1 mL methanol derived from potato, and then allowed to react at 37° C. for 17 hours.

After the reaction, 500 μL of hexane was added for extraction. The resulting hexane layer was concentrated to dryness with centrifugal evaporator. The dried material was redissolved into 10 μL of hexane, and all amount of the solution was spotted onto a TLC plate (Merck, TLC glass plate RP-18F254S 5×10 cm). Spots developed by acetone:water=19:1 was subjected to light exposure on an imaging plate and visualized using a scanner (Typhoon FLA9500).

### Comparative Example 1 (Negative Control)

The same operations were performed as in Example described above except that the proteoliposome was not added.

### Comparative Examples 2 to 9 (Other than Negative Control)

Plasmids for cell-free synthesis of the proteins were prepared to 1 μg/μL. The total amount of the plasmid in the reaction was 13.5 μL, and each plasmid was added in the same amount. Thereafter, the same operations were performed as in Example described above.

TABLE 1
Combination of protein and measurement result for radioactivity in Examples

| | CPT1 | CPT2 | CPT6 | CPT7 | CPTL | REF1 | REF2 | REF8 | SRPP1 | |
|---|---|---|---|---|---|---|---|---|---|---|
| | (C) | (D) | (A-1) | (A-2) | (B) | (E) | (F) | (G) | (H) | Radioactivity |
| Example 1 | − | − | + | − | + | − | − | − | − | 7655.5 |
| Example 2 | − | − | − | + | + | − | − | − | − | 5041.5 |
| Example 3 | + | + | + | + | + | + | + | + | + | 15173.2 |
| Example 4 | − | + | + | + | + | + | + | + | + | 9449.0 |
| Example 5 | + | − | + | + | + | + | + | + | + | 6764.0 |
| Example 6 | + | + | − | + | + | + | + | + | + | 2975.2 |
| Example 7 | + | + | + | − | + | + | + | + | + | 4660.0 |
| Example 8 | + | + | + | + | + | − | + | + | + | 8233.7 |
| Example 9 | + | + | + | + | + | + | − | + | + | 5697.5 |
| Example 10 | + | + | + | + | + | + | + | − | + | 7280.7 |
| Example 11 | + | + | + | + | + | + | + | + | − | 7143.3 |
| Comparative | − | − | − | − | − | − | − | − | − | 152.3 |
| example 1 | | | | | | | | | | |
| Comparative | + | − | − | − | − | − | − | − | − | 303.4 |
| example 2 | | | | | | | | | | |
| Comparative | − | + | − | − | − | − | − | − | − | 401.6 |
| example 3 | | | | | | | | | | |
| Comparative | − | − | + | − | − | − | − | − | − | 328.1 |
| example 4 | | | | | | | | | | |
| Comparative | − | − | − | + | − | − | − | − | − | 340.8 |
| example 5 | | | | | | | | | | |
| Comparative | + | − | − | − | + | − | − | − | − | 289.7 |
| example 6 | | | | | | | | | | |
| Comparative | + | − | − | − | + | − | + | − | − | 388.1 |
| example 7 | | | | | | | | | | |
| Comparative | + | + | + | + | − | + | + | + | + | 305.5 |
| example 8 | | | | | | | | | | |
| Comparative | + | + | − | − | + | + | + | + | + | 205.6 |
| example 9 | | | | | | | | | | |

As clearly demonstrated in Table 1, the radioactivity in each Example was higher than that in Comparative examples, revealing that the product (presumed to have a molecular weight of 1,000 to 2,000) was synthesized. The combination of CPT6 and CPTL in Example 1 and the combination of CPT7 and CPTL in Example 2 exhibit higher radioactivities than Comparative examples, demonstrating that each combination exhibits isoprene polymerization activity. Furthermore, other protein combinations in Examples 3 to 11 also exhibited higher radioactivities than Comparative examples. Some of them exhibited higher radioactivities than Examples 1 and 2, demonstrating that the proteins other than CPT6, 7, and CPTL are also capable of promoting the isoprene polymerization activity.

These results reveal that the present disclosure enables efficient production of natural rubber.

SEQ ID NO: 1: Nucleic acid sequence of CPT1

SEQ ID NO: 2: Amino acid sequence of CPT1

SEQ ID NO: 3: Nucleic acid sequence of CPT2

SEQ ID NO: 4: Amino acid sequence of CPT2

SEQ ID NO: 5: Nucleic acid sequence of CPT6

SEQ ID NO: 6: Amino acid sequence of CPT6

SEQ ID NO: 7: Nucleic acid sequence of CPT7

SEQ ID NO: 8: Amino acid sequence of CPT7

SEQ ID NO: 9: Nucleic acid sequence of CPTL

SEQ ID NO: 10: Amino acid sequence of CPTL

SEQ ID NO: 11: Nucleic acid sequence of REF1

SEQ ID NO: 12: Amino acid sequence of REF1

SEQ ID NO: 13: Nucleic acid sequence of REF2

SEQ ID NO: 14: Amino acid sequence of REF2

SEQ ID NO: 15: Nucleic acid sequence of REF8

SEQ ID NO: 16: Amino acid sequence of REF8

SEQ ID NO: 17: Nucleic acid sequence of SRPP1

SEQ ID NO: 18: Amino acid sequence of SRPP1

SEQ ID NO: 19: Primer 1 (for acquisition of CPT1 gene)

SEQ ID NO: 20: Primer 2 (for acquisition of CPT1 gene)

SEQ ID NO: 21: Primer 3 (for acquisition of CPT2 gene)

SEQ ID NO: 22: Primer 4 (for acquisition of CPT2 gene)

SEQ ID NO: 23: Primer 5 (for acquisition of CPT6 gene)

SEQ ID NO: 24: Primer 6 (for acquisition of CPT6 gene)

SEQ ID NO: 25: Primer 7 (for acquisition of CPT7 gene)

SEQ ID NO: 26: Primer 8 (for acquisition of CPT7 gene)

SEQ ID NO: 27: Primer 9 (for acquisition of CPTL gene)

SEQ ID NO: 28: Primer 10 (for acquisition of CPTL gene)

SEQ ID NO: 29: Primer 11 (for acquisition of REF1 gene)

SEQ ID NO: 30: Primer 12 (for acquisition of REF1 gene)

SEQ ID NO: 31: Primer 13 (for acquisition of REF2 gene)

SEQ ID NO: 32: Primer 14 (for acquisition of REF2 gene)

SEQ ID NO: 33: Primer 15 (for acquisition of REF8 gene)

SEQ ID NO: 34: Primer 16 (for acquisition of REF8 gene)

SEQ ID NO: 35: Primer 17 (for acquisition of SRPP1 gene)

SEQ ID NO: 36: Primer 18 (for acquisition of SRPP1 gene)

1. A protein composition, the protein composition comprising a protein(s) (B) and either one of proteins (A-1) or (A-2):
(A-1) one or more protein(s) selected from a protein(s) comprising an amino acid sequence of SEQ ID NO: 6 and a protein(s) that comprises an amino acid sequence having 90% or more identity with the amino acid sequence of SEQ ID NO: 6 and exhibits the same activity as CPT6;
(A-2) one or more protein(s) selected from a protein(s) comprising an amino acid sequence of SEQ ID NO: 8 and a protein(s) that comprises an amino acid sequence having 90% or more identity with the amino acid sequence of SEQ ID NO: 8 and exhibits the same activity as CPT7; and
(B) one or more protein(s) selected from a protein(s) comprising an amino acid sequence of SEQ ID NO: 10 and a protein(s) that comprises an amino acid sequence having 90% or more identity with the amino acid sequence of SEQ ID NO: 10 and exhibits the same activity as CPTL,
wherein the protein composition exhibits an isoprene polymerization activity.

2. The protein composition according to claim 1, wherein the protein composition fulfills at least one of the followings:
the protein (A-1) encoded by one or more polynucleotide(s) (a-1) selected from a polynucleotide(s) comprising a nucleic acid sequence of SEQ ID NO: 5 and a polynucleotide(s) that comprises a nucleic acid sequence having 90% or more identity with the nucleic acid sequence of SEQ ID NO: 5 and encodes a protein exhibiting the same activity as CPT6;
the protein (A-2) encoded by one or more polynucleotide(s) (a-2) selected from a polynucleotide(s) comprising a nucleic acid sequence of SEQ ID NO: 7 and a polynucleotide(s) that comprises a nucleic acid sequence having 90% or more identity with the nucleic acid sequence of SEQ ID NO: 7 and encodes a protein exhibiting the same activity as CPT7; or
the protein (B) encoded by one or more polynucleotide(s) (b) selected from a polynucleotide(s) comprising a nucleic acid sequence of SEQ ID NO: 9 and a polynucleotide(s) that comprises a nucleic acid sequence having 90% or more identity with the nucleic acid sequence of SEQ ID NO: 9 and encodes a protein exhibiting the same activity as CPTL.

3. The protein composition according to claim 1, further comprising an REF family protein, an SRPP family protein, and/or a CPT family protein other than CPT6, 7 and CPTL.

4. The protein composition according to claim 1, further comprising at least one of the followings:
(C) one or more protein(s) selected from a protein(s) comprising an amino acid sequence of SEQ ID NO: 2 and a protein(s) that comprises an amino acid sequence having 90% or more identity with the amino acid sequence of SEQ ID NO: 2 and exhibits the same activity as CPT1;
(D) one or more protein(s) selected from a protein(s) comprising an amino acid sequence of SEQ ID NO: 4 and a protein(s) that comprises an amino acid sequence having 90% or more identity with the amino acid sequence of SEQ ID NO: 4 and exhibits the same activity as CPT2;
(E) one or more protein(s) selected from a protein(s) comprising an amino acid sequence of SEQ ID NO: 12 and a protein(s) that comprises an amino acid sequence having 90% or more identity with the amino acid sequence of SEQ ID NO: 12 and exhibits the same activity as REF1;
(F) one or more protein(s) selected from a protein(s) comprising an amino acid sequence of SEQ ID NO: 14 and a protein(s) that comprises an amino acid sequence having 90% or more identity with the amino acid sequence of SEQ ID NO: 14 and exhibits the same activity as REF2;
(G) one or more protein(s) selected from a protein(s) comprising an amino acid sequence of SEQ ID NO: 16 and a protein(s) that comprises an amino acid sequence having 90% or more identity with the amino acid sequence of SEQ ID NO: 16 and exhibits the same activity as REF8; or
(H) one or more protein(s) selected from a protein(s) comprising an amino acid sequence of SEQ ID NO: 18 and a protein(s) that comprises an amino acid sequence having 90% or more identity with the amino acid sequence of SEQ ID NO: 18 and exhibits the same activity as SRPP1.

5. The protein composition according to claim 4, wherein the protein composition fulfills at least one of the followings:
the protein (C) encoded by one or more polynucleotide(s) (c) selected from a polynucleotide(s) comprising a nucleic acid sequence of SEQ ID NO: 1 and a polynucleotide(s) that comprises a nucleic acid sequence having 90% or more identity with the nucleic acid sequence of SEQ ID NO: 1 and encodes a protein exhibiting the same activity as CPT1;
the protein (D) encoded by one or more polynucleotide(s) (d) selected from a polynucleotide(s) comprising a nucleic acid sequence of SEQ ID NO: 3 and a polynucleotide(s) that comprises a nucleic acid sequence having 90% or more identity with the nucleic acid sequence of SEQ ID NO: 3 and encodes a protein exhibiting the same activity as CPT2;
the protein (E) encoded by one or more polynucleotide(s) (e) selected from a polynucleotide(s) comprising a nucleic acid sequence of SEQ ID NO: 11 and a polynucleotide(s) that comprises a nucleic acid sequence having 90% or more identity with the nucleic acid sequence of SEQ ID NO: 11 and encodes a protein exhibiting the same activity as REF1;
the protein (F) encoded by one or more polynucleotide(s) (f) selected from a polynucleotide(s) comprising a nucleic acid sequence of SEQ ID NO: 13 and a polynucleotide(s) that comprises a nucleic acid sequence having 90% or more identity with the nucleic acid sequence of SEQ ID NO: 13 and encodes a protein exhibiting the same activity as REF2;
the protein (G) encoded by one or more polynucleotide(s) (g) selected from a polynucleotide(s) comprising a nucleic acid sequence of SEQ ID NO: 15 and a polynucleotide(s) that comprises a nucleic acid sequence having 90% or more identity with the nucleic acid sequence of SEQ ID NO: 15 and encodes a protein exhibiting the same activity as REF8; or
the protein (H) encoded by one or more polynucleotide(s) (h) selected from a polynucleotide(s) comprising a nucleic acid sequence of SEQ ID NO: 17 and a polynucleotide(s) that comprises a nucleic acid sequence having 90% or more identity with the nucleic acid sequence of SEQ ID NO: 17 and encodes a protein exhibiting the same activity as SRPP1.

6. The protein composition according to claim 4, wherein at least one of the proteins (A) through (H) is a protein derived from a rubber tree.

7. A lipid membrane structure comprising the protein composition according to claim 1 and a phospholipid.

8. The lipid membrane structure according to claim 7, wherein the lipid membrane structure is a lipid bilayered membrane structure.

9. The structure according to claim 7, wherein the lipid membrane structure is a proteoliposome.

10. A cell expressing a protein constituting the protein composition of claim 1.

11. The cell according to claim 10, comprising an expression unit containing a polynucleotide that encodes each of the proteins in the protein composition.

12. The cell according to claim 10, wherein the polynucleotide comprises (b) and either one of (a-1) or (a-2):
(a-1) one or more polynucleotide(s) selected from a polynucleotide(s) comprising a nucleic acid sequence of SEQ ID NO: 5 and a polynucleotide(s) that comprises a nucleic acid sequence having 90% or more identity with the nucleic acid sequence of SEQ ID NO: 5 and encodes a protein exhibiting the same activity as CPT6;
(a-2) one or more polynucleotide(s) selected from a polynucleotide(s) comprising a nucleic acid sequence of SEQ ID NO: 7 and a polynucleotide(s) that comprises a nucleic acid sequence having 90% or more identity with the nucleic acid sequence of SEQ ID NO: 7 and encodes a protein exhibiting the same activity as CPT7; and
(b) one or more polynucleotide(s) selected from a polynucleotide(s) comprising a nucleic acid sequence of SEQ ID NO: 9 and a polynucleotide(s) that comprises a nucleic acid sequence having 90% or more identity with the nucleic acid sequence of SEQ ID NO: 9 and encodes a protein exhibiting the same activity as CPTL.

13. The cell according to claim 10, wherein the polynucleotide is the polynucleotide encoding the protein constituting the protein composition, and further comprises at least one of the followings:
(c) one or more polynucleotide(s) selected from a polynucleotide(s) comprising a nucleic acid sequence of SEQ ID NO: 1 and a polynucleotide(s) that comprises a nucleic acid sequence having 90% or more identity with the nucleic acid sequence of SEQ ID NO: 1 and encodes a protein exhibiting the same activity as CPT1;
(d) one or more polynucleotide(s) selected from a polynucleotide(s) comprising a nucleic acid sequence of SEQ ID NO: 3 and a polynucleotide(s) that comprises a nucleic acid sequence having 90% or more identity with the nucleic acid sequence of SEQ ID NO: 3 and encodes a protein exhibiting the same activity as CPT2;
(e) one or more polynucleotide(s) selected from a polynucleotide(s) comprising a nucleic acid sequence of SEQ ID NO: 11 and a polynucleotide(s) that comprises a nucleic acid sequence having 90% or more identity with the nucleic acid sequence of SEQ ID NO: 11 and encodes a protein exhibiting the same activity as REF1;
(f) one or more polynucleotide(s) selected from a polynucleotide(s) comprising a nucleic acid sequence of SEQ ID NO: 13 and a polynucleotide(s) that comprises a nucleic acid sequence having 90% or more identity with the nucleic acid sequence of SEQ ID NO: 13 and encodes a protein exhibiting the same activity as REF2;
(g) one or more polynucleotide(s) selected from a polynucleotide(s) comprising a nucleic acid sequence of SEQ ID NO: 15 and a polynucleotide(s) that comprises a nucleic acid sequence having 90% or more identity with the nucleic acid sequence of SEQ ID NO: 15 and encodes a protein exhibiting the same activity as REF8; or
(h) one or more polynucleotide(s) selected from a polynucleotide(s) comprising a nucleic acid sequence of SEQ ID NO: 17 and a polynucleotide(s) that comprises a nucleic acid sequence having 90% or more identity with the nucleic acid sequence of SEQ ID NO: 17 and encodes a protein exhibiting the same activity as SRPP1.

14. The cell according to claim 10, wherein the expression unit is a heterologous expression unit.

15. The cell according to claim 10, comprising a lipid membrane structure, which is a proteoliposome comprising the protein composition and a phospholipid.

16. A method for producing an isoprene polymer compound, the method comprising performing an isoprene polymerization reaction using at least one selected from the protein composition according to claim 1.

17. The production method according to claim 16, wherein the isoprene polymerization reaction is performed using a low molecular weight allyl compound as a substrate.

18. A method for producing a rubber, the method comprising producing the rubber using an isoprene polymer compound produced by the production method according to claim 16.


SEQUENCE LISTING (36 sequences)

SEQ ID NO: 1 - DNA, 873 bp, Hevea brasiliensis
  source 1..873: mol_type = genomic DNA
atggaattatacaacggtgagaggccaagtgtgttcagacttttagggaagtatatgagaaaagggttatatagcatcctaacccagggtcccatccctactcatattgccttcatattggatggaaacaggaggtttgctaagaagcataaactgccagaaggaggtggtcataaggctggatttttagctcttctgaacgtactaacttattgctatgagttaggagtgaaatatgcgactatctatgcctttagcatcgataattttcgaaggaaacctcatgaggttcagtacgtaatggatctaatgctggagaagattgaagggatgatcatggaagaaagtatcatcaatgcatatgatatttgcgtacgttttgtgggtaacctgaagcttttaagtgagccagtcaagaccgcagcagataagattatgagggctactgccaacaattccaaatgtgtgcttctcattgctgtatgctatacttcaactgatgagatcgtgcatgctgttgaagaatcctctgaattgaactccaatgaagtttgtaacaatcaagaattggaggaggcaaatgcaactggaagcggtactgtgattcaaattgagaacatggagtcgtattctggaataaaacttgtagaccttgagaaaaacacctacataaatccttatcctgatgttctgattcgaacttctggggagacccgtctgagcaactacttactttggcagactactaattgcatactgtattctcctcatgcactgtggccagagattggtcttcgacacgtggtgtgggcagtaattaactgccaacgtcattattcttacttggagaaacataaggaatacttaaaataa

SEQ ID NO: 2 - protein, 290 aa, Hevea brasiliensis
MELYNGERPSVFRLLGKYMRKGLYSILTQGPIPTHIAFILDGNRRFAKKHKLPEGGGHKAGFLALLNVLTYCYELGVKYATIYAFSIDNFRRKPHEVQYVMDLMLEKIEGMIMEESIINAYDICVRFVGNLKLLSEPVKTAADKIMRATANNSKCVLLIAVCYTSTDEIVHAVEESSELNSNEVCNNQELEEANATGSGTVIQIENMESYSGIKLVDLEKNTYINPYPDVLIRTSGETRLSNYLLWQTTNCILYSPHALWPEIGLRHVVWAVINCQRHYSYLEKHKEYLK

SEQ ID NO: 3 - DNA, 891 bp, Hevea brasiliensis
  source 1..891: mol_type = genomic DNA
atggaaatatatacgggtcagaggccaagtgtgtttagaatttttgggaaatacatgagaaaagggttatatagcatcctaacccaaggtcccatccctactcatcttgccttcataatggatggaaaccggaggtttgctaagaagcacaaaatgaaagaagcagaaggttataaggcaggatatttagctcttctgagaacactaacttattgctatgagttgggagtgaggtatgtaaccatttatgcctttagcattgataattttcgaaggcaacctcgtgaggttcagtgcgtaatgaatctaatgatggagaagattgaagagattatcgtggaagaaagtatcatgaatgcatatgatgttggcgtacgtattgtgggtaacctgaatcttttagatgagccaatcaggatcgcagcagaaaagattatgagggctactgccaataattccaggtttgtgcttctcattgctgtagcctatagttcaactgatgagatcgtgcatgctgttgaagaatcctctaaagacaaattgaactccaatgaagtttgcaacaatgggattgaagctgaacaagaatttaaggaggcaaacggaactggaaacagtgtgattccagttcagaagacggagtcatattctggaataaatcttgcagaccttgagaaaaacacctacgtaaatcctcatcctgatgtcttgattcgaacttctgggttgagccgtctaagtaactacctactttggcagactagtaattgcatactgtattctccttttgcactgtggccagagattggtctcaggcacttggtatggacagtaatgaacttccaacgtcatcattcttatttggagaagcataaggaatatttaaaataa

SEQ ID NO: 4 - protein, 296 aa, Hevea brasiliensis
MEIYTGQRPSVFRIFGKYMRKGLYSILTQGPIPTHLAFIMDGNRRFAKKHKMKEAEGYKAGYLALLRTLTYCYELGVRYVTIYAFSIDNFRRQPREVQCVMNLMMEKIEEIIVEESIMNAYDVGVRIVGNLNLLDEPIRIAAEKIMRATANNSRFVLLIAVAYSSTDEIVHAVEESSKDKLNSNEVCNNGIEAEQEFKEANGTGNSVIPVQKTESYSGINLADLEKNTYVNPHPDVLIRTSGLSRLSNYLLWQTSNCILYSPFALWPEIGLRHLVWTVMNFQRHHSYLEKHKEYLK

SEQ ID NO: 5 - DNA, 1122 bp, Hevea brasiliensis
  source 1..1122: mol_type = genomic DNA
atggaaaaacatagcagtagtagagtgagtgagctgtttggaaatttgggtagttttattagaatttgcatatttcgtgtgttatccatgggacccatccccaatcattttgccttcataatggatggaaatcggaggtatgctaagaaggagaacatgaaaaaaggggcaggtcatagggctggattcttagctcttatatccatacttaagtactgctatgaattgggagtgaagtatgtaacaatttacgcctttagcattgataatttcaaaaggagtcctgatgaagttaaggacctgatggatctgatgctagaaaagattgaggatttgctgagggacgaaagcattgtgaaccaatatggaatcagagtatattttataggtaatttgaaacttttgagtgaaactgtgaggattgcagcagaaaaggttatgaaagctactgccaaaaacaccaattgtacccttttaatctgcgtagcctatacgtcacgtgatgagattgtacatgctgttcaagtttcatgtaaaaataaacaggaggaaattcaaccattgagcttttgtaaagctaataatgatgccattgaagaagtagaggataataagaaggttaatggagtcatcccatttgtttttttagaatcccagaaagatgaagcaggcaaatctcaagcaacaatggcaagtgtaacctgcagttgtctggctagaggagttgaagggggtggcaacaaaaatagcatggttgttcgtgctgtccgaggatcctatgaagataaatgggataactgtcaagcaatgatggaaaatagaactggcaatggtgtgactttatctgaagagagtgagaatatgcagggagagtgttctatcataaagctagtagacattgagaaacagatgtacatggctgtagctcctgaacctgacatccttattcgaagttctggagagtcccgcctgagtaacttcctactttggcagaccagtgagtgccagttatattctccagatgcattgtggccagaaattggtttatggcacttggtgtgggcagtattaaacttccaacgaaaccatgcttatttggaaaagaaaaagcatcagttataa

SEQ ID NO: 6 - protein, 373 aa, Hevea brasiliensis
MEKHSSSRVSELFGNLGSFIRICIFRVLSMGPIPNHFAFIMDGNRRYAKKENMKKGAGHRAGFLALISILKYCYELGVKYVTIYAFSIDNFKRSPDEVKDLMDLMLEKIEDLLRDESIVNQYGIRVYFIGNLKLLSETVRIAAEKVMKATAKNTNCTLLICVAYTSRDEIVHAVQVSCKNKQEEIQPLSFCKANNDAIEEVEDNKKVNGVIPFVFLESQKDEAGKSQATMASVTCSCLARGVEGGGNKNSMVVRAVRGSYEDKWDNCQAMMENRTGNGVTLSEESENMQGECSIIKLVDIEKQMYMAVAPEPDILIRSSGESRLSNFLLWQTSECQLYSPDALWPEIGLWHLVWAVLNFQRNHAYLEKKKHQL

SEQ ID NO: 7 - DNA, 939 bp, Hevea brasiliensis
  source 1..939: mol_type = genomic DNA
atgcaatccttgcacttggccctccctctccctttttacaacactctaactcctcaaaaacacaaacacttcctttctcccgatgccagaactaatacacgcccaattcaccgccttctcggggccaaaacagacgtagctgtcaaacaggaggaaactattgctgtaagcaccggagactcgcccaagactgaacctttgccggaggggctccggggagagttgatgccacggcatgtcgccgtgatcatggacggcaatgggaggtgggcccagctgcgaggtcagatggcatcgatgggtcatcaggctggtgcacggtctttgctggagatcgtgcagctttcttgtcaatgggggattaaagttcttagcgtttttgcgttttcttgcgataattggactaggcccaaagtcgtgcaggtggagattgatttcttgatgagtttgttcgaaagcgtgttaaagtcagagatggataaatttgtgagggaaggtattcgaatctctgtgatcggggactcatcaaggcttccacagtctttgcaaagattaataaatgaagtggaggagaccaccagaaatttctcgaaactgcaccttctagtggcggttagctacagtggaaagtatgatgttgtaaaagcatgcaaaagtattgcttgtctggtaaaggatggtgttattgaaccagaagacattagcgaaagcctaattgagcaggagttggaaacaaattgctccgagtttccctcccctgatttattaatccgaactagtggtgaacttagaattagcaacttcttgctatggcagttggcctacactgaacttttctttgcggaagaactctggcctgattttggaaaaactggattcatagaggccttaacttcataccaacaaaggcaaagacgctatggtagacgacttacataa

SEQ ID NO: 8 - protein, 312 aa, Hevea brasiliensis
MQSLHLALPLPFYNTLTPQKHKHFLSPDARTNTRPIHRLLGAKTDVAVKQEETIAVSTGDSPKTEPLPEGLRGELMPRHVAVIMDGNGRWAQLRGQMASMGHQAGARSLLEIVQLSCQWGIKVLSVFAFSCDNWTRPKVVQVEIDFLMSLFESVLKSEMDKFVREGIRISVIGDSSRLPQSLQRLINEVEETTRNFSKLHLLVAVSYSGKYDVVKACKSIACLVKDGVIEPEDISESLIEQELETNCSEFPSPDLLIRTSGELRISNFLLWQLAYTELFFAEELWPDFGKTGFIEALTSYQQRQRRYGRRLT

SEQ ID NO: 9 - DNA, 774 bp, Hevea brasiliensis
  source 1..774: mol_type = genomic DNA
atggatttgaaacctggagctggagggcagagagttaatcgtttagtggatccgattagttatcattttcttcaatttctgtggcgtactctacatcttcttgtcagcttatggtaccttcaagttagtatggtccaaatgatcgaaggctttctaatctctagtggacttgtgaaacgctatggagccctcgatattgacaaggtccggtaccttgccattgtggtagatagtgaagaagcttaccaaatttctaaagttattcagcttttgaaatgggtggaagatatgggtgtgaaacatttatgcctctatgattcaaaaggagttctcaagacaaacaagaaaaccatcatggagagtttgaacaatgctatgccatttgaggaagcagttgaaaaagatgttttactggaccagaaacagatgactgtggaatttgcttccagctccgatggaaaggaagcaataaccagggcagctaacgtactctttatgaagtatttgaagtatgctaaaactggtgtaggaaaggaagaaccatgctttacagaagatcaaatggatgaggcactaaaagctataggttacaaagggccggaacctgacttgctattaatttatggacctgttagatgccatctaggtttctcaccgtggagacttcgatatactgagatggtgcatatgggacccttgaggtacatgaacctcggttcactaaaaaaggccattcacaggttcacaacagtgcagcaaaattatggtacataa

SEQ ID NO: 10 - protein, 257 aa, Hevea brasiliensis
MDLKPGAGGQRVNRLVDPISYHFLQFLWRTLHLLVSLWYLQVSMVQMIEGFLISSGLVKRYGALDIDKVRYLAIVVDSEEAYQISKVIQLLKWVEDMGVKHLCLYDSKGVLKTNKKTIMESLNNAMPFEEAVEKDVLLDQKQMTVEFASSSDGKEAITRAANVLFMKYLKYAKTGVGKEEPCFTEDQMDEALKAIGYKGPEPDLLLIYGPVRCHLGFSPWRLRYTEMVHMGPLRYMNLGSLKKAIHRFTTVQQNYGT

SEQ ID NO: 11 - DNA, 528 bp, Hevea brasiliensis
  source 1..528: mol_type = genomic DNA
atggctgaaggtgaagaagaggtgaatatccaagaagaggcaaataaaggagaggagaatccccaagaagaggcgaatatccaagaagagacgaataaaggagaggagaatatccaagaagaggcgaatatcgaagaagaggctaataaggaggaagagagcctaaagtatttggattttgtgcaagcggctacagtttatgccagggcttctttctcaaagctctacctttttgccaaggacaagtctggtccattcaagcctggcgtcaatactgttgagagtaggtttaagagcgtggttagacccgtctataataagttccaacctgttcccaacaaggttctcaagtttgcagaccgtagggttgatgcatatgtcactgttttagatcgcattgttcctccaattgtcaagcgggcatctatccaagcttattcagtagccccaggagctgctcgtgctgtggcttcttatttgcctttgcataccaagagactttttaaggtgctatatggagatgggtga

SEQ ID NO: 12 - protein, 175 aa, Hevea brasiliensis
MAEGEEEVNIQEEANKGEENPQEEANIQEETNKGEENIQEEANIEEEANKEEESLKYLDFVQAATVYARASFSKLYLFAKDKSGPFKPGVNTVESRFKSVVRPVYNKFQPVPNKVLKFADRRVDAYVTVLDRIVPPIVKRASIQAYSVAPGAARAVASYLPLHTKRLFKVLYGDG

SEQ ID NO: 13 - DNA, 417 bp, Hevea brasiliensis
  source 1..417: mol_type = genomic DNA
atggctgaagacgaagacaaccaacaagggcagggggaggggttaaaatatttgggttttgtgcaagacgcggcaacttatgctgtgactaccttctcaaacgtctatctttttgccaaagacaaatctggtccactgcagcctggtgtcgatatcattgagggtccggtgaagaacgtggctgtacctctctataataggttcagttatattcccaatggagctctcaagtttgtagacagcacggttgttgcatctgtcactattatagatcgctctcttcccccaattgtcaaggacgcatctatccaagttgtttcagcaattcgagctgccccagaagctgctcgttctctggcttcttctttgcctgggcagaccaagatacttgctaaggtgttttatggagagaattga

SEQ ID NO: 14 - protein, 138 aa, Hevea brasiliensis
MAEDEDNQQGQGEGLKYLGFVQDAATYAVTTFSNVYLFAKDKSGPLQPGVDIIEGPVKNVAVPLYNRFSYIPNGALKFVDSTVVASVTIIDRSLPPIVKDASIQVVSAIRAAPEAARSLASSLPGQTKILAKVFYGEN

SEQ ID NO: 15 - DNA, 354 bp, Hevea brasiliensis
  source 1..354: mol_type = genomic DNA
atggctgaagggaaagaaaacgagaatttccaacaagaggctaatgagcaggaagagaagctaaagtatttggaatttgtacaagcgactacagataatgctgtaactgccctctcaaacatttacctttatgccaaggacaattctggtccgttgaagcctggggtcgagactattgagggtgtggcaaagaccgtggttattccggccagtaaaattcccactgaagctatcaagtttgcagacagagcggtggatgcatctttcactactctacaaaacattgttccctcagttctcaagcagttgcctacccaagcttgcgatactagtgtgaaggaaagtgcagagtaa

SEQ ID NO: 16 - protein, 117 aa, Hevea brasiliensis
MAEGKENENFQQEANEQEEKLKYLEFVQATTDNAVTALSNIYLYAKDNSGPLKPGVETIEGVAKTVVIPASKIPTEAIKFADRAVDASFTTLQNIVPSVLKQLPTQACDTSVKESAE

SEQ ID NO: 17 - DNA, 615 bp, Hevea brasiliensis
  source 1..615: mol_type = genomic DNA
atggctgaagaggtggaggaagagaggctaaagtatttggattttgtgcgagcggctggagtttatgctgtagattctttctcaactctctacctttatgccaaggacatatctggtccattaaaacctggtgtcgatactattgagaatgtggtgaagaccgtggttactcctgtttattatattccccttgaggctgtcaagtttgtagacaaaacggtggatgtatcggtcactagcctagatggcgttgttcccccagttatcaagcaggtgtctgcccaaacttactcggtagctcaagatgctccaagaattgttcttgatgtggcttcttcagttttcaacactggtgtgcaggaaggcgcaaaagctctgtacgctaatcttgaaccaaaagctgagcaatatgcggtcattacctggcgtgccctcaataagctgccactagttcctcaagtggcaaatgtagttgtgccaaccgctgtttatttctctgaaaagtacaacgatgttgttcgtggcactactgagcagggatatagagtgtcctcttatttgcctttgttgcccactgagaaaattactaaggtgtttggagatgaggcatcataa

SEQ ID NO: 18 - protein, 204 aa, Hevea brasiliensis
MAEEVEEERLKYLDFVRAAGVYAVDSFSTLYLYAKDISGPLKPGVDTIENVVKTVVTPVYYIPLEAVKFVDKTVDVSVTSLDGVVPPVIKQVSAQTYSVAQDAPRIVLDVASSVFNTGVQEGAKALYANLEPKAEQYAVITWRALNKLPLVPQVANVVVPTAVYFSEKYNDVVRGTTEQGYRVSSYLPLLPTEKITKVFGDEAS

SEQ ID NO: 19 - DNA, 19 bp, synthetic construct
  misc_feature 1..19: note = Primer 1 for amplifying CPT1
  source 1..19: mol_type = other DNA
atggaattatacaacggtg

SEQ ID NO: 20 - DNA, 36 bp, synthetic construct
  misc_feature 1..36: note = Primer 2 for amplifying CPT1
  source 1..36: mol_type = other DNA
atctcgagttaatgatgatgatgatgatgttttaag

SEQ ID NO: 21 - DNA, 21 bp, synthetic construct
  misc_feature 1..21: note = Primer 3 for amplifying CPT2
  source 1..21: mol_type = other DNA
atggaaatatatacgggtcag

SEQ ID NO: 22 - DNA, 41 bp, synthetic construct
  misc_feature 1..41: note = Primer 4 for amplifying CPT2
  source 1..41: mol_type = other DNA
atctcgagttaatgatgatgatgatgatgttttaaatattc

SEQ ID NO: 23 - DNA, 20 bp, synthetic construct
  misc_feature 1..20: note = Primer 5 for amplifying CPT6
  source 1..20: mol_type = other DNA
atggaaaaacatagcagtag

SEQ ID NO: 24 - DNA, 29 bp, synthetic construct
  misc_feature 1..29: note = Primer 6 for amplifying CPT6
  source 1..29: mol_type = other DNA
atctcgagttatataactgatgctttttc

SEQ ID NO: 25 - DNA, 18 bp, synthetic construct
  misc_feature 1..18: note = Primer 7 for amplifying CPT7
  source 1..18: mol_type = other DNA
atgcaatccttgcacttg

SEQ ID NO: 26 - DNA, 30 bp, synthetic construct
  misc_feature 1..30: note = Primer 8 for amplifying CPT7
  source 1..30: mol_type = other DNA
atctcgagttatgtaagtcgtctaccatag

SEQ ID NO: 27 - DNA, 17 bp, synthetic construct
  misc_feature 1..17: note = Primer 9 for amplifying CPTL
  source 1..17: mol_type = other DNA
atggatttgaaacctgg

SEQ ID NO: 28 - DNA, 34 bp, synthetic construct
  misc_feature 1..34: note = Primer 10 for amplifying CPTL
  source 1..34: mol_type = other DNA
atctcgagttaatgatgatgatgatgatgtgtac

SEQ ID NO: 29 - DNA, 30 bp, synthetic construct
  misc_feature 1..30: note = Primer 11 for amplifying REF1
  source 1..30: mol_type = other DNA
atggctgaaggtgaagaagaggtgaatatc

SEQ ID NO: 30 - DNA, 29 bp, synthetic construct
  misc_feature 1..29: note = Primer 12 for amplifying REF1
  source 1..29: mol_type = other DNA
atctcgagtcacccatctccatatagcac

SEQ ID NO: 31 - DNA, 28 bp, synthetic construct
  misc_feature 1..28: note = Primer 13 for amplifying REF2
  source 1..28: mol_type = other DNA
atggctgaagacgaagacaaccaacaag

SEQ ID NO: 32 - DNA, 29 bp, synthetic construct
  misc_feature 1..29: note = Primer 14 for amplifying REF2
  source 1..29: mol_type = other DNA
atctcgagtcaattctctccataaaacac

SEQ ID NO: 33 - DNA, 30 bp, synthetic construct
  misc_feature 1..30: note = Primer 15 for amplifying REF8
  source 1..30: mol_type = other DNA
atggctgaagggaaagaaaacgagaatttc

SEQ ID NO: 34 - DNA, 29 bp, synthetic construct
  misc_feature 1..29: note = Primer 16 for amplifying REF8
  source 1..29: mol_type = other DNA
atctcgagttactctgcactttccttcac

SEQ ID NO: 35 - DNA, 28 bp, synthetic construct
  misc_feature 1..28: note = Primer 17 for amplifying SRPP1
  source 1..28: mol_type = other DNA
atggctgaagaggtggaggaagagaggc

SEQ ID NO: 36 - DNA, 30 bp, synthetic construct
  misc_feature 1..30: note = Primer 18 for amplifying SRPP1
  source 1..30: mol_type = other DNA
atctcgagttatgatgcctcatctccaaac